(19) United States
(12) Patent Application Publication
(10) Pub. No.: US 2026/0240873 A1
(43) Pub. Date: Aug. 20, 2026

(54) USE OF CANNABIDIOL IN THE TREATMENT OF TUBEROUS SCLEROSIS COMPLEX

(71) Applicant: GW Research Limited, Cambridge (GB)

(72) Inventors: Geoffrey GUY, Cambridge (GB); Volker KNAPPERTZ, Cambridge (GB); Eduardo DUNAYEVICH, Carlsbad, CA (US); Daniel CHECKETTS, Cambridge (GB)

(21) Appl. No.: 17/606,370

(22) PCT Filed: May 1, 2020
(86) PCT No.: PCT/GB2020/051080

§ 371 Date: Oct. 25, 2021

(30) Foreign Application Priority Data
May 3, 2019 (GB) 1906261.1

(51) Int. Cl. A61K 31/05; A61K 31/197; A61K 31/20; A61K 31/4015; A61K 31/5513; A61K 36/185; A61P 25/08

(52) U.S. Cl. CPC A61K 31/658; A61K 31/197; A61K 31/20; A61K 31/4015; A61K 31/5513; A61K 36/3482; A61P 25/08

(57) ABSTRACT

The present invention relates to the use of a cannabidiol (CBD) preparation for use in the treatment of seizures associated with tuberous sclerosis complex (TSC). Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used. In use the CBD is given concomitantly with one or more other anti-epileptic drugs (AED). Alternatively, the CBD may be formulated for administration separately, sequentially or simultaneously with one or more AED or the combination may be provided in a single dosage form.

### FIELD OF THE INVENTION

The present invention relates to the use of a cannabidiol (CBD) preparation for use in the treatment of seizures associated with tuberous sclerosis complex (TSC).

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

In use the CBD is given concomitantly with one or more other anti-epileptic drugs (AED). Alternatively, the CBD may be formulated for administration separately, sequentially or simultaneously with one or more AED or the combination may be provided in a single dosage form.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Whilst focal seizures with impairment are a type of seizure commonly found to occur in the epileptic syndrome tuberous sclerosis complex (TSC) these patients experience a range of different seizure types. TSC is a genetic disorder that causes mainly benign tumours to develop in certain parts of the body. When tumours develop in the brain these often cause seizures, which are often localized in one area of the brain where the tumour is.

Epilepsy is a very common feature of TSC however many patients suffering from seizures associated with TSC are unable to obtain control of their seizures using existing AED. Alternative treatments such as surgery to remove the tumours in the brain or vagus nerve stimulation may be helpful.

Epileptic syndromes such as TSC often present with many different types of seizure. Identifying the types of seizure that a patient is suffering from is important as many of the standard AED's are targeted to treat a given seizure type these can be both generalised and focal seizure types.

Over the past forty years there have been a number of animal and human studies on the use of the non-psychoactive cannabinoid cannabidiol (CBD) to treat seizures.

A study in 1978 provided 200 mg/day of pure CBD to four adult patients, two of the four patients became seizure free, whereas in the remainder, seizure frequency was unchanged (Mechoulam and Carlini, 1978).

Cunha et al. reported that administration of CBD to eight adult patients with generalized epilepsy resulted in a marked reduction of seizures in 4 of the patients (Cunha et al., 1980) and Consroe et al., (1982) determined that CBD was able to prevent seizures in mice after administration of pro-convulsant drugs or an electric current.

In contrast to the studies described above, an open label study reported that 200 mg/day of pure CBD was ineffective in controlling seizures in twelve institutionalized adult patients (Ames and Cridland, 1986).

All of the studies described above focused on the treating subjects suffering from generalised epilepsy and did not look at the treatment of specific seizure sub-types.

The patent applications WO 2011/001169 describes the use of CBD in the treatment of focal seizures; WO 2012/093255 describes the use of CBD in combination with standard anti-epileptic drugs in the treatment of epilepsy; and WO 2013/045891 describes a composition comprising CBD and CBDV for use in the treatment of epilepsy.

Porter and Jacobson (2013) report on a parent survey conducted via a Facebook group which explored the use of cannabis which was enriched with CBD in children with treatment-resistant epilepsy. It was found that sixteen of the 19 parents surveyed reported an improvement in their child's epilepsy. The children surveyed for this paper were all taking cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported. Providing children with TRE with a cannabis extract that comprises THC, which has been described as a pro-convulsant (Consroe et al., 1977), at a potentially psychoactive dose of 0.8 mg/kg/day, is a concern.

An open label study demonstrated the use of CBD in the treatment of refractory epilepsy in the treatment of 10 patients with Tuberous Sclerosis Complex (Geffrey et al., 2014). In addition, WO 2016/059399 describes the use of CBD in the treatment of TSC at doses which were titrated up to 25 mg/kg/day.

The applicant has found by way of a double-blind placebo-controlled trial that doses of 25 and 50 mg/kg/day of CBD resulted in a statistically significant reduction in seizures associated with TSC.

### BRIEF SUMMARY OF THE DISCLOSURE

In accordance with a first aspect of the present invention there is provided a cannabidiol (CBD) preparation for use in the treatment of seizures associated with tuberous sclerosis complex (TSC).

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the CBD preparation is used in combination with one or more concomitant anti-epileptic drugs (AED).

More preferably the one or more AED is selected from the group consisting of: valproic acid, vigabatrin, levetiracetam and clobazam.

In one embodiment the CBD is present is isolated from cannabis plant material. Preferably at least a portion of at least one of the cannabinoids present in the CBD preparation is isolated from cannabis plant material.

In a further embodiment the CBD is present as a synthetic preparation. Preferably at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In accordance with a second aspect of the present invention there is provided a method of treating a patient suffering from seizures associated with tuberous sclerosis complex (TSC) comprising administering a cannabidiol (CBD) preparation to the subject in need thereof.

Seizures that are associated with TSC include one or more of the following seizure types: focal motor seizures without impairment of consciousness or awareness; focal seizures with impairment of consciousness or awareness; focal seizures evolving to bilateral generalized convulsive seizures and generalized seizures (tonic-clonic, tonic, clonic or atonic) that are countable.

### Definitions

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“TSC-associated seizure” is defined as one or more of the following seizure types: focal motor seizures without impairment of consciousness or awareness; focal seizures with impairment of consciousness or awareness; focal seizures evolving to bilateral generalized convulsive seizures and generalized seizures (tonic-clonic, tonic, clonic or atonic) that are countable.

“Focal Seizures” are defined as seizures which originate within networks limited to only one hemisphere. What happens during the seizure depends on where in the brain the seizure happens and what that part of the brain normally does.

“Focal seizure with impairment” has replaced the term “complex partial seizure”. These seizures usually start in a small area of the temporal lobe or frontal lobe of the brain and involve other areas of the brain within the same hemisphere that affect alertness and awareness. Most subjects experience automatisms during a focal seizure with impaired consciousness.

“Mixed seizures” are defined as the existence of both generalised and focal seizures in the same patient.

The terms “50% responder” and “50% reduction in seizure” are both terms used in clinical studies. In the present application the terms define the percentage of subjects that experienced a greater than or equal to 50% reduction in the total number of seizures during treatment with CBD in comparison to the number experienced during the baseline period before the CBD was administered.

### DETAILED DESCRIPTION

### Preparation of Botanically Derived Purified Cbd

The following describes the production of the botanically derived purified CBD which comprises greater than or equal to 98% w/w CBD and less than or equal to other cannabinoids was used in the placebo-controlled trial described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/W.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification |
|---|---|---|
| Identification: | Visual | Complies |
| A | TLC | Corresponds to standard |
| B | HPLC/UV | (for CBD & CBDA) |
| C | | Positive for CBDA |
| Assay: | In-house | NLT 90% of assayed |
| CBDA + CBD | (HPLC/UV) | cannabinoids by peak area |
| Loss on Drying | Ph. Eur. | NMT 15% |
| Aflatoxin | UKAS method | NMT 4 ppb |
| Microbial: | Ph. Eur. | NMT10⁷ cfu/g |
| TVC | | NMT10⁵ cfu/g |
| Fungi | | NMT10² cfu/g |
| E. coli | | |
| Foreign Matter: | Ph. Eur. | NMT 2% |
| Residual Herbicides and | Ph. Eur. | Complies |
| Pesticides | | |

TABLE B
Specification of an exemplary botanically
derived purified CBD preparation

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass |
| | | spectrum of major peak |
| | | corresponds to certified |
| | | CBD Reference Standard |
| Identification C | FT-IR | Conforms to reference spectrum |
| | | for certified CBD Reference |
| | | Standard |
| Identification D | Melting Point | 65-67° C. |
| Identification E | Specific Optical | Conforms with certified CBD |
| | Rotation | Reference Standard; −110° |
| | | to −140° (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | GC | NMT 0.5% w/w |
| Alkane | | NMT 0.5% w/w |
| Ethanol | | |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a) Growing
b) Direct drying
c) Decarboxylation
d) Extraction-using liquid CO₂
e) Winterization using ethanol
f) Filtration
g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a) Crystallization using C₅-C₁₂ straight chain or branched alkane
b) Filtration
c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in a double-blind, randomized, placebo-controlled study to investigate the efficacy and safety of CBD as an add-on therapy in patients with tuberous sclerosis complex (TSC) who experience inadequately-controlled seizures.

### Example 1: Efficacy of Cannabidiol Reducing Seizures in Children and Young Adults with Tuberous Sclerosis Complex

### Study Design

The blinded phase of the study was a randomized, double-blind, parallel-group, 16-week comparison of two doses of CBD versus placebo. Patients completed a 1-week screening period and a 4-week baseline period before they were randomized to receive 25 mg/kg/day CBD, 50 mg/kg/day CBD or equivalent volumes of placebo. Randomization was stratified by age according to the following ranges: 1-6, 7-11, 12-17 years and 18+ years.

Patients began a 4-week dose escalation period, titrating up to their assigned dose, before continuing on a stable dose of blinded investigational medicinal product (IMP) for 12 weeks.

Dose escalation for each patient was subject to the investigator's assessment of safety and tolerability. If a dose became poorly tolerated, the investigator may consider temporarily or permanently reducing the dose for the remainder of the study.

Clinic visits occurred for screening (Day-35), baseline (Day-28), randomization (Day 1) and, on Days 15 and 29 during titration and on Days 43, 57, 71 (telephone) and 85 until the end of treatment (Day 113). Safety telephone calls were completed every two days during titration, one week after the end of titration and, for patients not entering the open label extension (OLE), weekly from Visit 10 to Visit 12.

Patients were required to perform daily interactive voice response system (IVRS) telephone calls to record seizure information. They were also required to complete a paper diary daily with information about IMP and concomitant AED administration.

Following completion of the blinded phase, patients were invited to continue to receive CBD in an OLE.

Those patients opting not to enter the OLE completed a 10-day taper period (down-titrating 10% per day for 10 days).

The OLE consisted of a 3-week titration period followed by a maintenance period and a 10-day taper period. The initial OLE period will last for a maximum of 1 year.

Following titration according to the titration schedule, patients will continue with their optimal CBD dose. However, investigators may decrease the dose if a patient experiences intolerance or increase the dose to a maximum of 50 mg/kg/day if required for better seizure control, until the optimal dose was found.

The primary endpoint was the change in number of TSC-associated seizures during the treatment period (maintenance and titration) compared to baseline in patients taking CBD compared with placebo.

TSC-associated seizures included: focal motor seizures without impairment of consciousness or awareness; focal seizures with impairment of consciousness or awareness; focal seizures evolving to bilateral generalized convulsive seizures and generalized seizures (tonic-clonic, tonic, clonic or atonic) that are countable.

Secondary endpoints included the number of patients considered treatment responders which was defined as those with a >50% reduction in TSC-associated seizure frequency;

change in Caregiver Global Impression of Change (CGIC) or Subject Global Impression of Change (SGIC) score; and change in total seizures.

Antiepileptic efficacy measures were also determined and these included: number of patients considered treatment responders defined as those with a ≥25%, ≥50%, ≥75% or 100% reduction in TSC-associated seizure frequency; number of patients experiencing a >25% worsening, −25 to +25% no change, 25-50% improvement, 50-75% improvement or >75% improvement in TSC-associated seizure frequency; change in number of TSC-associated seizure-free days; and change in number of ‘other’ seizures (absence, myoclonic, focal sensory and infantile/epileptic spasms).

Growth and development measures were undertaken in patients less than 18 years old. These included change in serum insulin-like growth factor-1 (IGF-1) levels and change in Tanner Staging score (for patients aged 10-17 [inclusive]).

Quality of life measures also recorded, these included changes in the Quality of Life in Childhood Epilepsy (QOLCE; patients 2-18 years) or Quality of Life in Epilepsy (QOLIE-31-P; patients 19+ years) score; change in Physician Global Impression of Change (PGIC) score.

Safety and tolerability measures that were recorded included: adverse events; clinical laboratory parameters; 12-lead electrocardiogram (ECG); physical examination parameters (including height and weight); vital signs; Columbia-Suicide Severity Rating Scale (C-SSRS; 19+ years) or C-SSRS Children's (6-18 years) score, where applicable; number of inpatient hospitalizations due to epilepsy; abuse liability and effects on menstruation cycles (in females).

### Patient Disposition, Demographic and Baseline Characteristics

A total of 201 patients completed the study, being made up of 75 placebo; 65 in the CBD 25 mg/kg/day treatment group and 61 in the CBD 50 mg/kg/day treatment group. Of those that completed 199 patients continued onto the OLE phase of the study.

There were slightly more males (58.5%) than females in the study and the majority of patients were white/Caucasian (89.7%). The mean age of trial participants was 13.6 years of age; however, the minimum age of patients was 1.14 and the eldest patient to participate was 56.8 years of age.

At baseline the median number of TSC-associated seizures the trial patients experienced was 56.93 over the 28-day baseline period, with a minimum of 7.7 and a maximum of 558. Table 1.1 below describes the baseline seizure rates.

TABLE 1.1
Baseline seizure rates

| Number of | | CBD | CBD | |
|---|---|---|---|---|
| seizures over | | 25 mg/ | 50 mg/ | |
| baseline | Placebo | kg/day | kg/day | Total |
| period | (n = 76) | (n = 75) | (n = 73) | (n = 224) |
| TSC- | 54.05 | 56.00 | 61.00 | 56.93 |
| associated | | | | |
| seizures | | | | |
| Type 1 | 33 (43.4%) | 29 (38.7%) | 39 (53.4%) | 101 (45.1%) |
| (focal | | | | |
| without | | | | |
| impairment) | | | | |
| Type 2 | 50 (65.8%) | 46 (61.3%) | 54 (74.0%) | 150 (67.0%) |
| (focal | | | | |
| with | | | | |
| impairment) | | | | |
| Type 3 | 24 (31.6%) | 17 (22.7%) | 24 (32.9%) | 65 (29.0%) |
| (focal | | | | |
| secondary | | | | |
| generalised) | | | | |
| Tonic-clonic | 14 (18.4%) | 22 (29.3%) | 16 (21.9%) | 52 (23.2%) |
| Tonic | 15 (19.7%) | 27 (36.0%) | 23 (31.5%) | 65 (29.0%) |
| Clonic | 2 (2.6%) | 3 (4.0%) | 3 (4.1%) | 8 (3.6%) |
| Atonic | 13 (17.1%) | 10 (13.3%) | 5 (6.8%) | 28 (12.5%) |
| Other | 15 (19.7%) | 12 (16.0%) | 24 (32.9%) | 51 (22.8%) |
| Total | 56.48 | 56.00 | 70.00 | 58.90 |

All patients were taking at least one anti-epileptic drug. The anti-epileptic drugs (AEDs) that the patients on the trial were taking were valproic acid; vigabatrin; levetiracetam; and clobazam. Table 1.2 below details the numbers of AEDs the patients were taking at baseline.

TABLE 1.2
Anti-epileptic drugs taken at baseline

| | | CBD | CBD | |
|---|---|---|---|---|
| | | 25 mg/ | 50 mg/ | |
| | Placebo | kg/day | kg/day | Total |
| | (n = 76) | (n = 75) | (n = 73) | (n = 224) |
| Median | 4 | 4 | 4 | 4 |
| number of | | | | |
| prior AEDs | | | | |
| Median | 3 | 3 | 3 | 3 |
| number of | | | | |
| current | | | | |
| AEDs | | | | |
| Valproic | 35 (46.1%) | 29 (38.7%) | 36 (49.3%) | 100 (44.6%) |
| acid | | | | |
| Vigabatrin | 35 (22.4%) | 28 (37.3%) | 29 (39.7%) | 74 (33.0%) |
| Levetir- | 24 (31.6%) | 19 (25.3%) | 22 (30.1%) | 65 (29.0%) |
| acetam | | | | |
| Clobazam | 25 (32.9%) | 17 (22.7%) | 19 (26.0%) | 61 (27.2%) |

### Results

Over the treatment period, (Day 1 to Day 113, 16 weeks), there was a significantly significant decrease in TSC-associated seizures in both CBD treatment groups as is shown in Table 1.3 below.

TABLE 1.3
TSC-associated seizures over the treatment period

| Mean number of | | | |
|---|---|---|---|
| seizures per 28 | Placebo | CBD 25 mg/kg/day | CBD 50 mg/kg/day |
| days | (n = 76) | (n = 75) | (n = 73) |
| Baseline period | 54.8 | 51.6 | 66.3 |
| Treatment period | 40.3 | 26.5 | 34.9 |

Table 1.4 provides the statistical analysis on these data.

TABLE 1.4
TSC-associated seizures over the treatment
period - statistical analysis

| Estimated ratio | | CBD 25 mg/ | CBD 50 mg/ |
|---|---|---|---|
| [percentage | Placebo | kg/day | kg/day |
| reduction] | (n = 76) | (n = 75) | (n = 73) |
| Treatment period/ | 0.735 [26.5%] | 0.514 [48.6%] | 0.525 [47.5%] |
| Baseline period | | | |
| CBD/Placebo | — | 0.699 [30.1%] | 0.715 [28.5%] |
| p-value | — | 0.0009 | 0.0018 |

In addition, over the maintenance period, which is defined as the 12-week period which does not include the 4-week titration period, there was a 30% reduction in TSC-associated seizures in the placebo group and a 55.8% reduction of seizures in the CBD (25 mg/kg/day) group (p=0.0004) and a 55.8% reduction of seizures in the CBD (50 mg/kg/day) group (p=0.0005).

Furthermore, there were 17 (22.4%) patients that received placebo who experienced a ≥50% reduction in seizures, 27 (36.0%) patients that received CBD (25 mg/kg/day) group (p=0.0692) and 29 (39.2%) patients that received CBD (50 mg/kg/day) group (p=0.0245). It was also found that 4 patients taking 25 mg/kg/day and 2 patients taking 50 mg/kg/day of CBD experienced complete seizure freedom, (100% reduction in TSC-associated seizure frequency).

Table 1.5 describes the subject/caregiver global impression of change in the trial.

### Table 1.5: Subject/Caregiver Global Impression of Change

TABLE 1.5
Subject/caregiver global impression of change

| | | CBD 25 mg/ | CBD 50 mg/ |
|---|---|---|---|
| | Placebo | kg/day | kg/day |
| Data from last visit | (n = 76) | (n = 75) | (n = 73) |
| Very much improved | 2 (2.8% | 10 (15.2%) | 9 (13.6%) |
| Much improved | 10 (13.9%) | 12 (18.2%) | 12 (18.2%) |
| Slightly improved | 15 (20.8%) | 23 (34.8%) | 21 (31.8%) |
| No change | 41 (56.9%) | 14 (21.2%) | 14 (21.2%) |
| Slightly worse | 3 (4.2%) | 4 (6.1%) | 6 (9.1%) |
| Much worse | 1 (1.4%) | 2 (3.0%) | 3 (4.5%) |
| Very much worse | 0 | 1 (1.5%) | 1 (1.5%) |
| p-value | — | 0.0074 | 0.0580 |

In addition to the TSC-associated seizures (focal motor seizures without impairment of consciousness or awareness; focal seizures with impairment of consciousness or awareness; focal seizures evolving to bilateral generalized convulsive seizures and generalized seizures (tonic-clonic, tonic, clonic or atonic) that are countable) the total seizure count was recorded in the trial. These data are detailed in Tables 1.6 and 1.7 below.

TABLE 1.6
Total seizure count during baseline and treatment period

| Mean number of | | | |
|---|---|---|---|
| seizures per | Placebo | CBD 25 mg/kg/day | CBD 50 mg/kg/day |
| 28 days | (n = 76) | (n = 75) | (n = 73) |
| Baseline period | 60.7 | 53.9 | 75.7 |
| Treatment period | 44.4 | 28.0 | 39.6 |

TABLE 1.7
Total seizures over the treatment period - statistical analysis

| Estimated ratio | | CBD 25 mg/ | CBD 50 mg/ |
|---|---|---|---|
| [percentage | Placebo | kg/day | kg/day |
| reduction] | (n = 76) | (n = 75) | (n = 73) |
| Treatment period/ | 0.731 [26.9%] | 0.519 [48.1%] | 0.524 [47.6%] |
| Baseline period | | | |
| CBD/Placebo | — | 0.709 [48.1%] | 0.716 [28.4%] |
| p-value | — | 0.0013 | 0.0018 |

In addition, over the maintenance period, which is defined as the 12-week period which does not include the 4-week titration period, there was a 30.1% reduction in TSC-associated seizures in the placebo group and a 55% reduction of seizures in the CBD (25 mg/kg/day) group (p=0.0007) and a 55.9% reduction of seizures in the CBD (50 mg/kg/day) group (p=0.0005).

The most common adverse reactions that occurred in patients receiving CBD in this study (≥10% and greater than placebo) included somnolence; decreased appetite; diarrhoea; vomiting; transaminase elevations; pyrexia; and infections. The incidence of diarrhoea, vomiting, transaminase elevations, somnolence, and rash were higher in the 50 mg/kg/day group as compared to the 25 mg/kg/day group.

In addition, 12% of patients in the 25 mg/kg/day group experienced alanine aminotransferase (ALT) elevations >3 upper level of normal (ULN) compared to 25% of patients in the 50 mg/kg/day group and none in patients on placebo. Patients on concomitant valproic acid and on the higher dose of CBD experienced a higher rate of ALT elevations and as such dose reductions in these treatment groups might be considered.

Overall treatment emergent adverse events were either mild or moderate as detailed in Table 1.8 below.

TABLE 1.8
Treatment emergent adverse event

| | | CBD 25 mg/ | CBD 50 mg/ |
|---|---|---|---|
| | Placebo | kg/day | kg/day |
| Maximal severity | (n = 76) | (n = 75) | (n = 73) |
| Mild | 48 (63.2%) | 37 (49.3%) | 29 (39.7%) |
| Moderate | 23 (30.3%) | 26 (34.7%) | 35 (47.9%) |
| Severe | 1 (1.3%) | 7 (9.3%) | 9 (12.3%) |

### CONCLUSIONS

These data indicate that CBD was able to significantly reduce the number of both TSC-associated seizures and the total number of seizures at both 25 and 50 mg/kg/day.

In the 25 mg/kg/day group there was additionally a significant efficacy in the subject/caregiver's global impression of change.

1-13. (canceled)

14. A method of treating a patient suffering from seizures associated with tuberous sclerosis complex (TSC) comprising administering a cannabidiol (CBD) drug substance to the subject in need thereof, wherein the CBD drug substance comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

15. The method of claim 14, wherein the CBD drug substance is used in combination with one or more concomitant anti-epileptic drugs (AED).

16. The method of claim 15, wherein the one or more AED is selected from the group consisting of: valproic acid, vigabatrin, levetiracetam and clobazam.

17. The method of claim 14, wherein the CBD is isolated from cannabis plant material.

18. The method of claim 14, wherein at least a portion of at least one of the cannabinoids present in the CBD preparation is isolated from cannabis plant material.

19. The method of claim 14, wherein the CBD is present as a synthetic preparation.

20. The method of claim 14, wherein at least a portion of at least one of the cannabinoids present in the CBD preparation is prepared synthetically.

21. The method of claim 14, wherein the dose of CBD is greater than 5 mg/kg/day.

22. The method of claim 14, wherein the dose of CBD is 20 mg/kg/day.

23. The method of claim 14, wherein the dose of CBD is 25 mg/kg/day.

24. The method of claim 14, wherein the dose of CBD is 50 mg/kg/day.

25. The method of claim 14, wherein the seizures that are associated with TSC comprise one or more of the following seizure types: focal motor seizures without impairment of consciousness or awareness; focal seizures with impairment of consciousness or awareness; or focal seizures evolving to bilateral generalized convulsive seizures and generalized seizures.
